(19) United States
(12) Patent Application Publication
(10) Pub. No.: US 2010/0262065 A1
(43) Pub. Date: Oct. 14, 2010

(54) Liquid submersed light emitting fiber optic handle apparatus system

(76) Inventor: Shane James Lynch, Livermore, CA (US)

Correspondence Address: Shane James Lynch, 1672 Honeysuckle Road, Livermore, CA, 94551, US

(21) Appl. No.: 12/384,922

(22) Filed: Apr. 9, 2009

(51) Int. Cl. A61B 18/18; A61M 1/00

(52) U.S. Cl. 604/20; 606/9

(57) ABSTRACT

This is an illuminating Liquid Submersed Light Emitting Fiber Optic Cable(s) which can use a variation of colors that are passed through the fiber optics and used in conjunction with a closed loop vacuum using a liquid or dry abrasion system for treatment to the surface of the skin. The illuminating light emitting color or multi color fiber optic cable(s) provides an on or pulse state of optic light that is submersed in a liquid and vacuum at the end of a replaceable end cap or tip. This is a safe way of transferring submersed fiber optic light with no electricity contact to the skin. The fiber optic light cable(s) are used in conjunction with a type of liquid that is passed or pulled through the handle apparatus. At the end of the handle apparatus is a replaceable end cap or tip.

This application claims priority of Provisional Application No. 61/044,727 dated Apr. 14, 2008.

### FIELD OF INVENTION

The present invention relates to skin care using a crystal free and liquid dermabrasion system and more specifically using a liquid submersed light emitting fiber optic cable or cables. The fiber optic cable or cables transmit light through the fiber optic(s) using a single or multiple light colors encased in a handle apparatus.

### BACKGROUND OF THE INVENTION

This process of treatment has been used for the improvement of the outer layer of the epidermis by removing dead skin cells from the top layer of the skin using a crystal free and liquid dermabrasion, which improves age spots, blemishes, fine lines, scarring and improves the appearance of dull skin. This is achieved using a device combined with vacuum, liquid flow passage ways and a stationary abrasive post at the end of the handle apparatus, which is contained inside of a disposable end cap or tip at the end of the handle apparatus.

The liquid submersed illuminating light emitting fiber optic(s) color or multi color fiber optic cables(s) are encased in the handle apparatus, which contain the vacuum, liquid flow passage ways and a stationary abrasive post and work in conjunction with the liquid submersed illuminating light emitting color or multi color fiber optic cable(s) for improving the skin's texture and appearance using a safe and effective method of dermabrasion and light therapy.

In spite of the advantages of the devices in prior art, there are some limitations which cannot be delivered using a liquid or crystal free dermabrasion without the use of the liquid submersed illuminating light emitting color or multi color fiber optic cable(s) in conjunction with a crystal free liquid dermabrasion process.

This invention improves conventional devices being used by incorporating the use of color or multi color submersed illuminating light emitting fiber optic cable(s) with liquid and vacuum passage ways along with a stationary abrasive post encased in the handle apparatus with disposable end cap or tip, creating a safe and effective method for improving the overall appearance of the skin.

### SUMMARY OF THE INVENTION

In accordance with the present invention, structures and associated methods to provide a crystal free liquid based applicator with illuminating light emitting fiber optic lighting. The applicator handle is using a vacuum, stationary abrasive post and liquid submersed illuminating light emitting fiber optic light source that in conjunction resurfaces the skin; improves skin tone and appearance, softens; scarring, fine wrinkles, acne and age spots. The illuminating light emitting fiber optic cable(s) are encased in the hand wand apparatus and improve the overall effectiveness of the dermabrasion treatment.

There are three separate containers; one which contains the clean liquid solution for the skin treatment, another container for collecting the used liquid and another container for flushing the system after usage.

The clean and used liquid containers and flushing container are kept separate from the manifold/junction block and handle assembly.

The outer tubing located at the hose fitting of the handle apparatus and the use of the inner hose fitting inside the handle apparatus allows the inner tubing, and fiber optic cable(s) to rotate inside of the handle apparatus freely with minimum binding.

This is a destination point where the outer tubing, inner tubing and fiber optic cable(s) meet inside of the manifold and then separated outside of the manifold to the following destination points:

Fiber opti—circuitry

Tubing manifold—liquid pump and pressure pump

### BRIEF DESCRIPTION OF THE DRAWINGS

The drawings constitute a part of this specification and include exemplary embodiments to this invention, which may be embodied in various forms. It is to be understood that in some instances various aspects of the invention may be shown exaggerated or enlarged to facilitate an understanding of the invention.

FIG. 1 is a schematic view of the invention in a preferred embodiment of a handle apparatus that encases liquid tubing, a vacuum source and the use of fiber optic cable(s) adapted in conjunction with a dermabrasion system.

FIG. 1. Is an illuminating light emitting fiber optic cable(s) that are used in conjunction with a closed loop pressure and vacuum liquid dermabrasion system for treatment to the surface of the skin. The illuminating light emitting fiber optic cable(s) provide a on or pulse state of optic light(s) that is submersed in a liquid or a dry vacuum at the end of the cap or tip. This is a safe way of transferring a submersed light source with no electrical contact to the skin. The illuminating light emitting fiber optic cables(s) are used in conjunction with a type of liquid that is passed or pulled through the handle apparatus. At the end of the handle apparatus is a replaceable end cap or tip.

FIG. 1. Clean liquids are pulled through tube 12 and passage way 09c and circulate in the end cap or tip 09b. Used liquids are pulled through vacuum passage way 11e and tube 13 to a used liquid container.

FIG. 2. Is a manifold/junction block 14a, which is a connection point for the fittings and tubing(s) and illuminating light emitting fiber optic cables(s).

### DETAILED DESCRIPTION OF THE INVENTION

The Illuminating Light Emitting single color or multiple colors fiber optic cable(s) for skin care of the present invention enables the light source to provide light in conjunction with a crystal free, liquid or liquid free dermabrasion directly to the skin's surface. Detailed descriptions of the preferred embodiment are provided herein. It is understood that the present invention may be embodied in various forms.

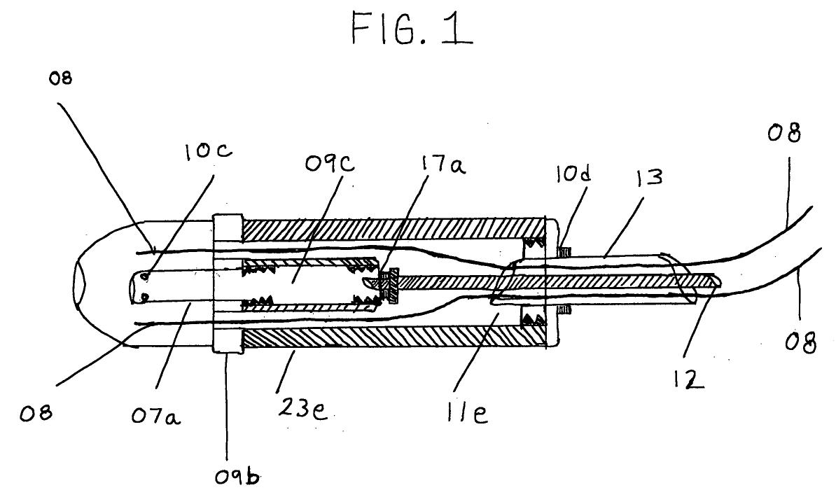

FIG. 1 A stationary abrasive hollow core attachment 07a with an upper top round radius covered with a type of textured finish. The stationary abrasive hollow core attachment is provided with one or two passageways 10c to allow fluid to be pushed or pulled through the removable end cap or tip 09b

FIG. 1 Within the removable end cap or tip 09b is a stationary abrasive hollow core attachment 07a, illuminating light emitting color or multiple color fiber optic cable(s) 08, which are submersed in a circulatory liquid or dry application inside of the removable end cap or tip 09b is connected to the handle apparatus 23e. The illuminating light emitting color or multi color fiber optic cable(s) 08 are located within the designated vacuum passage ways lie or can be placed in additional passageways, if desired.

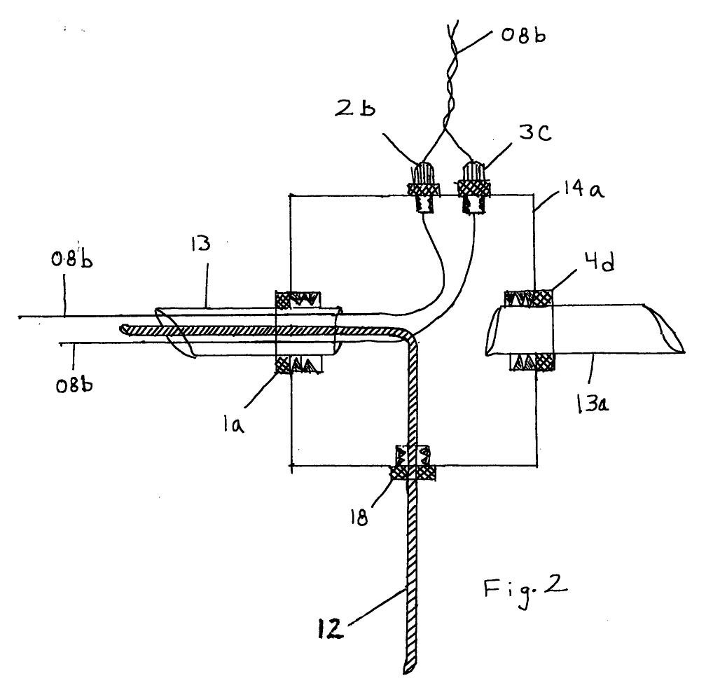

FIG. 1 The inner tube for liquid passage way 12 is located within the larger outer hose fitting 13. The clean liquid passage way tube 12 is connected to a hose fitting 17a at the clean liquid passage way 09c. This allows the light emitting color or multi color fiber optic cable(s) 08 and the clean liquid passage way inner tube 12 to rotate within the outer tube 13. The outer tube 13 is connected to a hose fitting 10d. The outer tube 13 with the clean liquid passage way inner tube 12 and illuminating light emitting color or multi color fiber optic cable(s) 08 locate themselves at FIG. 2 manifold 14a. Clean liquids are pulled through the clean liquid passage way 12 and passage way 09c and circulate in the removable end cap or tip 09b. Used liquids are pulled through vacuum passage ways 11e and hose tube 13 to a waste liquid container

FIG. 2 The Liquid Submersed Light Emitting Fiber Optic Cable(s) 08b are located within the manifold junction block 14a and are fed through the fittings 2b and 3c. The outer tubing 13 is installed at fitting 1a and stops within the manifold. The outer tube 13a is attached to fitting 4d. The tubing from fitting 4d is attached to a container for liquid waste. The clean liquid passage way tube 12 located is connected to fitting 18 at junction block and can be used as a liquid vacuum source or a dry vacuum source in conjunction with the manifold/junction block and operator handle apparatus system.

### BRIEF DESCRIPTION OF DRAWINGS

FIG. 1
07a Stationary abrasive hollow core attachment
08 Liquid Submersed Light Emitting Fiber Optic Handle Apparatus
09b Removable end cap or tip
09c Clean liquid passage way
10c Passageways in stationary abrasive hollow core attachment
10d Hose fitting
11e Designated vacuum passage way
12 Clean liquid passageway
13 Outer hose tube
14a Manifold junction block
17a Hose fittings
23e Handle Apparatus

FIG. 2
1a Hose fitting
2b Hose fitting
3c Hose fitting
4d Hose fitting
08b Illuminating color or multi color fiber optic cable(s)
12 Clean liquid passageway
13 Tubing
13a Tubing
14a Manifold junction block
Hose fitting

What I claim is:

1. A handle apparatus having an abrasive hollow core stand off post at the top of the handle with external and internal passages ports in conjunction with fiber optic cable or cables, fittings, connections and tubing in a closed loop vacuum system. The various color options for the fiber optic cable or cables are passed through one or more fiber optic cable(s) within the handle apparatus from the manifold junction block and electronic circuitry.

2. The arrangement of the tubing, fittings, connectors and fiber optic cable or cables, internally and externally at the manifold junction block, allows the tubing, fittings, connectors and fiber optic cable or cables to the operators handle apparatus to have a single outer hose and one internal hose with internal fiber optic cable or cables that work in conjunction in a closed loop vacuum system.

3. The operators handle apparatus is used in either two of the following functions: vacuum submersed liquid applications or a vacuum dry application. The operators handle apparatus is used with the light source turned offor the fiber optic light(s) source turned on from an electronic switch not shown in the illustration or drawings. The intensity of the fiber optic light(s) is controlled by a pulsed width modulation process contained in the electronic circuitry not shown in the illustration or drawing.
